(19) United States
(12) Patent Application Publication
(10) Pub. No.: US 2009/0204000 A1
(43) Pub. Date: Aug. 13, 2009

(54) ULTRASONIC DIAGNOSTIC APPARATUS

(76) Inventors: Yoko Okamura, Nasushiobara-shi (JP); Naohisa Kamiyama, Otawara-shi (JP); Tetsuya Yoshida, Nasushiobara-shi (JP); Yasuhiko Abe, Otawara-shi (JP); Tetsuya Kawagishi, Nasushiobara-shi (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 12/364,611

(22) Filed: Feb. 3, 2009

(30) Foreign Application Priority Data
Feb. 13, 2008 (JP) 2008-032253

(51) Int. Cl. A61B 8/14; A61B 8/00

(52) U.S. Cl. 600/443; 600/461

(57) ABSTRACT

An ultrasonic diagnostic apparatus includes an image creating unit configured to create an image of an affected area and an image of a puncture needle inserted into a subject by means of an ultrasonic scanning operation, and a monitor configured to display the image of the affected area and the image of the puncture needle created by the image creating unit, wherein the image creating unit displays a tissue expected to be extracted from the affected area as an image on the monitor before the puncture needle is inserted into the affected area.

### CROSS-REFERENCE TO RELATED APPLICATIONS

This application is based upon and claims the benefit of priority from prior Japanese Patent Application No. 2008-032253, filed Feb. 13, 2008, the entire contents of which are incorporated herein by reference.

### BACKGROUND OF THE INVENTION

1. Field of the Invention

The present invention relates to an ultrasonic diagnostic apparatus for assisting a puncture needle's extracting operation in which a tissue of an extracted portion is extracted.

2. Description of the Related Art

In a recent medical treatment, for example, when a cancer is suspected by means of an image diagnosis, a final decision is made by carrying out a needle biopsy in many cases. As a puncture needle for the needle biopsy, there is known a puncture needle formed in a double structure provided with an outer needle and an inner needle inserted into the outer needle. In a case where the needle biopsy is carried out by the puncture needle having the double structure, an affected area (extracted portion) to be extracted is set to a target, and the puncture needle is inserted into a position in the vicinity of the affected area. Subsequently, when a gun button is pressed, the inner needle instantly and automatically protrudes from the outer needle to extract a tissue thereof.

Meanwhile, since an ultrasonic diagnostic apparatus scans a subject using an ultrasonic wave and displays the ultrasonic image on a monitor, it is possible to see the affected area (extracted portion) as an image in real time. For this reason, the ultrasonic diagnostic apparatus is frequently used for the needle biopsy (for example, see Japanese Patent Application Laid-Open No. 2000-185041).

However, in the related art, since a portion on the image to be extracted upon pressing the gun button is just predicted by a doctor, precision during the needle biopsy deteriorates. For this reason, in a case where an examination result for the extracted tissue is largely different from the doctor's prediction, the needle biopsy needs to be carried out again, thereby causing a problem in that process efficiency deteriorates and a patient's burden increases.

Additionally, the doctor may check later a portion where the tissue is actually extracted by the needle biopsy.

However, in the related art, since the image of the tissue extracted by the needle biopsy is not stored as evidence, a problem arises in that the needle biopsy needs to be meaninglessly carried out again to check the image of the extracted tissue.

### BRIEF SUMMARY OF THE INVENTION

The present invention is contrived in consideration of the above-described circumstances, and an object of the invention is to provide an ultrasonic diagnostic apparatus capable of reliably extracting a tissue of a desired extracted portion and of storing an image of the extracted tissue as evidence after the tissue is extracted in actual.

According to an aspect of the invention, there is disclosed an ultrasonic diagnostic apparatus for two-dimensionally or three-dimensionally scanning a subject using an ultrasonic wave from an ultrasonic contact member and for assisting an operation for extracting a tissue of an extracted portion, detected by the ultrasonic scanning operation, using a puncture needle, the ultrasonic diagnostic apparatus including: an image creating device configured to create an image of the extracted portion and an image of the puncture needle inserted into the subject by means of the ultrasonic scanning operation; and a display device configured to display the image of the extracted portion and the image of the puncture needle created by the image creating device, wherein the image creating device displays a tissue expected to be extracted from the extracted portion on the display device before the puncture needle is inserted into the extracted portion.

According to another aspect of the invention, there is disclosed an ultrasonic diagnostic apparatus for two-dimensionally or three-dimensionally scanning a subject using an ultrasonic wave from an ultrasonic contact member and for assisting an operation for extracting a tissue of an extracted portion, detected by the ultrasonic scanning operation, using a puncture needle, the ultrasonic diagnostic apparatus including: an image creating device configured to create an image of the extracted portion and an image of the puncture needle inserted into the subject by means of the ultrasonic scanning operation; and a display device configured to display the image of the extracted portion and the image of the puncture needle created by the image creating device, wherein the image creating device displays the extracted tissue as an image on the display device and stores the image as evidence after the tissue of the extracted portion is extracted by the puncture needle.

According to the invention, since it is possible to predict a portion of the extracted portion to be extracted just by seeing the display device prior to the extraction of the tissue of the extracted portion, it is possible to reliably extract the desired tissue.

Further, since the image of the actually extracted tissue is stored as evidence, it is possible to easily check the image of the extracted tissue later on. Thus, it is not necessary to meaninglessly carry out the needle biopsy again.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing a configuration of an ultrasonic diagnostic apparatus according to an embodiment of the invention.

FIG. 2 is a perspective view showing a probe of the ultrasonic diagnostic apparatus shown in FIG. 1 and a puncture needle mounted to the probe.

FIG. 3 is a side sectional view showing the puncture needle shown in FIG. 2.

FIG. 4 is a view showing a 3D image of an affected area obtained by the ultrasonic diagnostic apparatus shown in FIG. 1.

FIG. 5 is a view showing an image of the puncture needle inserted toward an image of the affected area shown in FIG. 4.

FIG. 6A is a view showing a tomographic image of the puncture needle and the affected area in a surface where the puncture needle is inserted.

FIG. 6B is a view showing a tomographic image of the puncture needle and the affected area in a surface perpendicular to the surface where the puncture needle is inserted.

FIG. 6C is a view showing a tomographic image of the puncture needle and the affected area in a surface perpendicular to the surface where the puncture needle is inserted, the perpendicular surface being in a different direction from that of FIG. 6B.

FIG. 7A is a view showing an image of a tissue expected to be extracted in a state shown in FIG. 6A.

FIG. 7B is a view showing an image of a tissue expected to be extracted in a state shown in FIG. 6B.

FIG. 7C is a view showing an image of a tissue expected to be extracted in a state shown in FIG. 6C.

FIG. 7D is a view showing an image of a tissue expected to be extracted in a state shown in FIG. 5.

FIG. 8A is a view showing an image of a tissue actually extracted in a state shown in FIG. 6A.

FIG. 8B is a view showing an image of a tissue actually extracted in a state shown in FIG. 6B.

FIG. 8C is a view showing an image of a tissue actually extracted in a state shown in FIG. 6C.

FIG. 8D is a view showing an image of a tissue actually extracted in a state shown in FIG. 5.

FIG. 9 is a perspective view showing a probe of an ultrasonic diagnostic apparatus according to a second embodiment of the invention and a puncture needle mounted to the probe.

FIG. 10 is a view showing a tomographic image of an affected area in a surface where the puncture needle is inserted.

FIG. 11 is a view showing an image of the puncture needle inserted toward the image of the affected area shown in FIG. 10.

FIG. 12 is a view showing an image of a tissue expected to be extracted in a case where an inner needle of the puncture needle is assumed to protrude in a state shown in FIG. 11.

FIG. 13 is a view showing an image of a tissue actually extracted in a case where the inner needle of the puncture needle protrudes in a state shown in FIG. 11.

### DETAILED DESCRIPTION OF THE INVENTION

Hereinafter, exemplary embodiments of the invention will be described in detail with reference to the accompanying drawings.

### First Embodiment

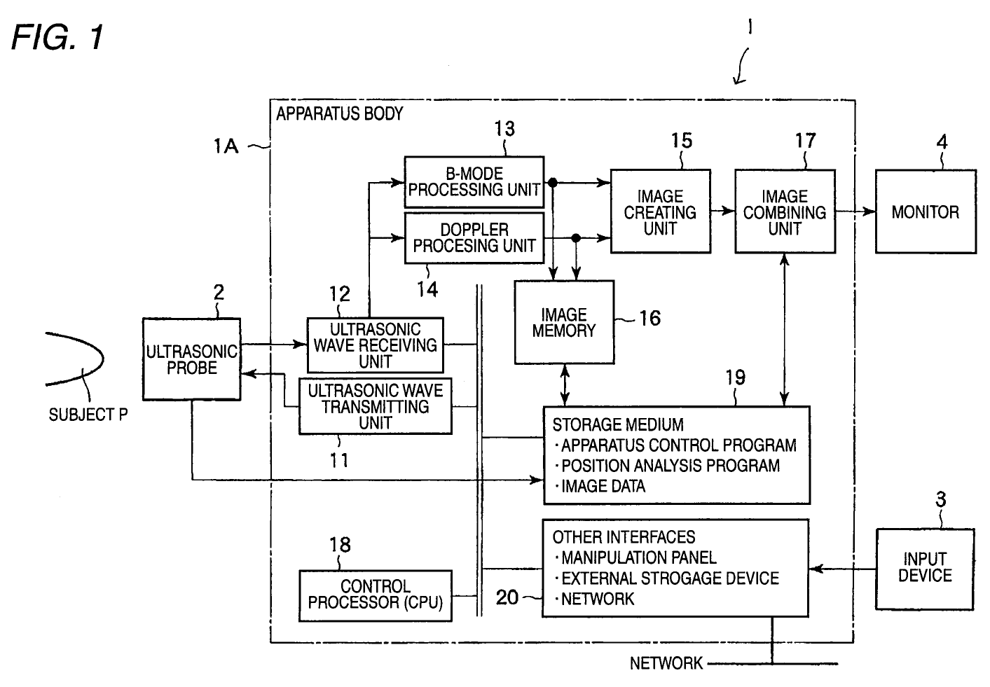

FIG. 1 is a block diagram showing a configuration of an ultrasonic diagnostic apparatus 1 according to an embodiment of the invention.

The ultrasonic diagnostic apparatus 1 includes an apparatus body 1A, a probe 2 as an ultrasonic contact member, an input device 3, and a monitor 4 as a display device.

The apparatus body 1A includes therein an ultrasonic wave transmitting unit 11, an ultrasonic wave receiving unit 12, a B-mode processing unit 13, a Doppler processing unit 14, an image creating unit 15 as an image creating device, an image memory 16, an image combining unit 17, a control processor (CPU) 18, an internal storage unit 19, an interface unit 20, and an optical sensor (not shown).

The probe 2 generates an ultrasonic wave on the basis of a driving signal output from the ultrasonic wave transmitting unit 11 and transmits the ultrasonic wave to a subject P. The probe 2 includes a plurality of piezoelectric vibrators for converting a wave reflected from the subject P into an electric signal, a matching layer formed in the piezoelectric vibrators, a packing material for preventing the ultrasonic wave from being transmitted backward from the piezoelectric vibrators, and the like.

When the ultrasonic wave is transmitted from the probe 2 to the subject P, the transmitted ultrasonic wave is continuously reflected in a discontinuous surface of an acoustic impedance of a body tissue and the probe 2 receives the reflected ultrasonic wave in a form of an echo signal. Amplitude of the echo signal is dependent on an acoustic impedance difference in the discontinuous surface where the reflection of the ultrasonic wave occurs. Additionally, in a case where the transmitted ultrasonic pulse is reflected in a surface of a moving bloodstream, a cardiac wall, or the like, the echo is dependent on a speed component of a mobile object in an ultrasonic transmission direction due to a Doppler effect and a frequency shift occurs. Additionally, position information of the probe 2 is transmitted to an on-demand storage unit 19 together with a collected data. The input device 3 is connected to the apparatus body 1A, and includes various switches, a button, a trackball, a mouse, a keyboard, and the like used to input various instructions, an instruction for setting a condition and an region of interest (ROI), and an instruction for setting various image quality conditions from an operator to the apparatus body 1A. When the operator manipulates an end button or a freeze button of the input device 3, the ultrasonic wave transmitting-receiving operation ends, and the ultrasonic diagnostic apparatus is in a pause state.

The monitor 4 displays a variety of information described below as an image.

The ultrasonic wave transmitting unit 11 includes a trigger generating circuit, a delay circuit, a pulser circuit, and the like which are not shown in the drawings. The pulser circuit repeatedly generates a rate pulse for forming a transmission ultrasonic wave at a predetermined rate frequency frHz (cycle; l/fr second). Additionally, the delay circuit gives a delay time necessary for collecting the ultrasonic wave in a beam shape for each channel and determining transmitting directivity to each rate pulse. The trigger generating circuit applies a drive pulse to the probe 2 at a timing based on the rate pulse.

Additionally, the ultrasonic wave transmitting unit 11 has a function of instantly changing a transmission frequency, a transmission driving voltage, and the like in order to carry out a predetermined scanning sequence in accordance with an instruction of the control processor 18. Particularly, the change of the transmission driving voltage is realized by a linear amp type generating circuit capable of instantly changing the value or a mechanism capable of electrically changing a plurality of power source units.

The ultrasonic wave receiving unit 12 includes an amp circuit, an A/D converter, an adder, and the like which are not shown in the drawings. The amp circuit amplifies the echo signal input from the probe 2 for each channel. The A/D converter gives a delay time necessary for determining receiving directivity for the amplified echo signal, and carries out an adding process by the adder. In terms of the adding process, a reflection component is emphasized in a direction in accordance with the receiving directivity of the echo signal, and a synthetic beam for transmitting or receiving the ultrasonic wave is formed by the receiving directivity and the transmitting directivity.

The B-mode processing unit 13 receives the echo signal from the ultrasonic wave transmitting unit 11, carries out a logarithmic amplifying process, an envelope detection process, and the like, and then creates a data in which signal strength is expressed by brightness.

The Doppler processing unit 14 carries out a frequency analysis of a speed component from the echo signal received from the ultrasonic wave transmitting unit 11, extracts an echo component of a bloodstream, a tissue, or a visualizing agent by the Doppler effect, and then obtains bloodstream information such as an average speed, dispersion, and a power at multiple points. Additionally, the Doppler processing unit 14 is configured to recognize a movement of an inner needle 24 protruding from an outer needle 23 of a puncture needle 22 as described below.

The image creating unit 15 displays a data signal sent from the B-mode processing unit 13 on the monitor 4 in a form of a B-mode image in which strength of a reflected wave is expressed by brightness. At this time, various image filters such as an edge emphasis, a time smoothing, and a space smoothing are carried out, thereby providing an image quality in accordance with a user's taste. Additionally, the image creating unit 15 color-displays the bloodstream information sent from the Doppler processing unit 14 on the monitor 4 in a form of an average speed image, a dispersion image, a power image, or a combined image thereof. Further, the image creating unit 15 converts a scanning line signal array for the ultrasonic scanning operation into a scanning signal array of a general video format represented as a television, and creates an ultrasonic diagnostic image as a display image.

Additionally, the image creating unit 15 is equipped with a storage memory for storing an image data so that the user calls an image stored during an examination after the diagnosis. A data which is not yet input to the image creating unit 15 is referred to as “a raw data” in some cases.

The image memory 16 is, for example, a memory for storing ultrasonic images corresponding to multiple frames just before a freezing operation. It is possible to play an ultrasonic video clip in such a manner that the images stored in the image memory 16 are continuously displayed (cine display).

The image combining unit 17 combines the image received from the image creating unit 15 with a scale, text information of various parameters, or the like, and outputs the combined image to the monitor 4 in a form of a video signal.

The control processor 18 has a function of an information processing device (calculator), and is a control device for controlling an operation of the present ultrasonic diagnostic apparatus body 1A. The control processor 18 reads out a control program, used for carrying out an image creating-displaying operation, from the internal storage unit 19, and executes the control program on its memory, thereby carrying out a calculation-control operation and the like of various processes.

The internal storage unit 19 stores a control program used for carrying out a transmitting-receiving condition, an image creation, and a display process; diagnostic information (a patient ID, a doctor comment, and the like); a diagnostic protocol; probe position information; a body mark creating program; and other data groups. Additionally, if necessary, the internal storage unit 19 is used for image storage of the image memory 16. The internal storage unit 19 is capable of transmitting its data to an external peripheral device via the interface unit 20.

The interface unit 20 is an interface used for the input device 3, a network, and a new external storage device (not shown). The interface unit 20 is capable of transmitting a data such as an ultrasonic image or an analysis result obtained by the apparatus to other devices via the network.

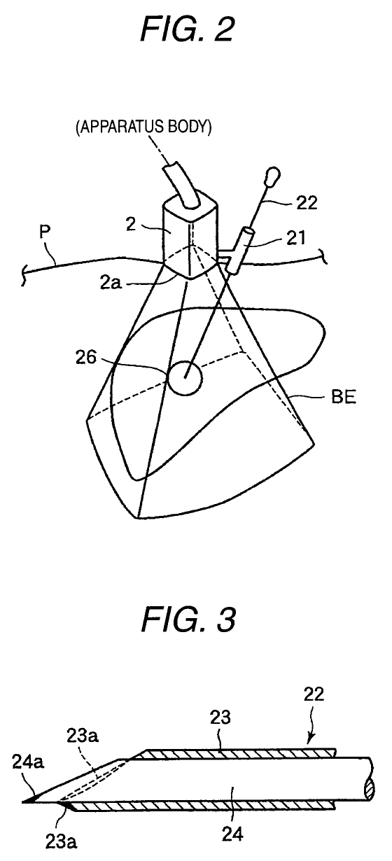

Incidentally, the probe 2 is mounted with a guiding puncture adaptor 21 as shown in FIG. 2, and the puncture needle 22 is inserted in the puncture adaptor 21 so as to advance or recede.

Additionally, the probe 2 has, for example, a configuration in which a plurality of ultrasonic vibrators is arranged in a two-dimension shape. When each ultrasonic vibrator is driven in a predetermined three-dimension scanning condition by a driving mechanism (not shown), an ultrasonic beam BE is three-dimensionally operated from a vibrator surface (vibrator arrangement surface) 2a to an affected area (extracted portion) 26 in the subject P. That is, a three-dimension volume scanning operation is carried out, and an echo signal of the ultrasonic wave is converted into an echo signal of a minute voltage in accordance with its strength, thereby detecting the echo signal and transmitting the echo signal to the apparatus body 1A.

Further, the echo signal, transmitted from the probe 2 to the apparatus body 1A, is transmitted to the B-mode processing unit 13 and the Doppler processing unit 14 via the ultrasonic wave receiving unit 12 in a form of a data, and the data is transmitted to the image creating unit 15. The image creating unit 15 creates an ultrasonic image (3D image) on the basis of the data, and creates an image of the puncture needle 22 inserted into a biological body during a needle biopsy described below. Additionally, the image creating unit 15 creates a tomographic image in a surface where the puncture needle 22 is inserted and creates tomographic images in surfaces perpendicular to the puncture-needle-insertion surface in two directions. Further, as described below, when the inner needle 24 is assumed to protrude from the outer needle 23 in a state where a front end portion of the puncture needle 22 inserted into the biological body arrives at a position in the vicinity of the affected area 26, the image creating unit 15 is capable of forming an image of the tissue at the affected area 26 expected to be extracted.

The image of the tissue at the affected area 26 expected to be extracted is created on the basis of the protruding amount of the inner needle 24 of the puncture needle 22 described below.

Meanwhile, the puncture needle 22 is formed in a double structure as shown in FIG. 3. That is, the puncture needle 22 includes the tubular outer needle 23 and the bar-shaped inner needle 24 slidably inserted into the outer needle 23. The outer needle 23 and the inner needle 24 are made of stainless steel or the like, and the front end portion is provided with blades 23a and 24a. The puncture needle 22 includes a gun button (not shown). When the gun button is pressed, the inner needle 24 protrudes from the outer needle 23 to be inserted into the affected area 26 and to extract a tissue thereof. The protruding amount of the inner needle 24 from the outer needle 23 is set to a predetermined amount.

Additionally, the puncture adaptor 21 is provided with a position sensor (not shown) or a rotary roller (not shown) rotating on the basis of the inserted amount of the puncture needle 22 so as to recognize the front end position by measuring the inserted distance of the puncture needle 22.

Next, a case will be described in which the needle biopsy is carried out by means of the ultrasonic diagnostic apparatus described above.

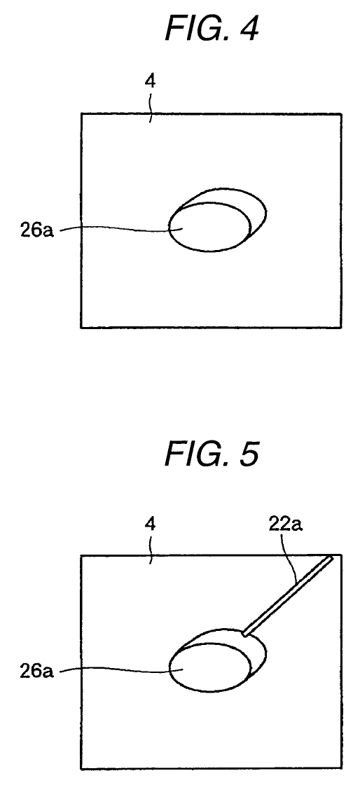

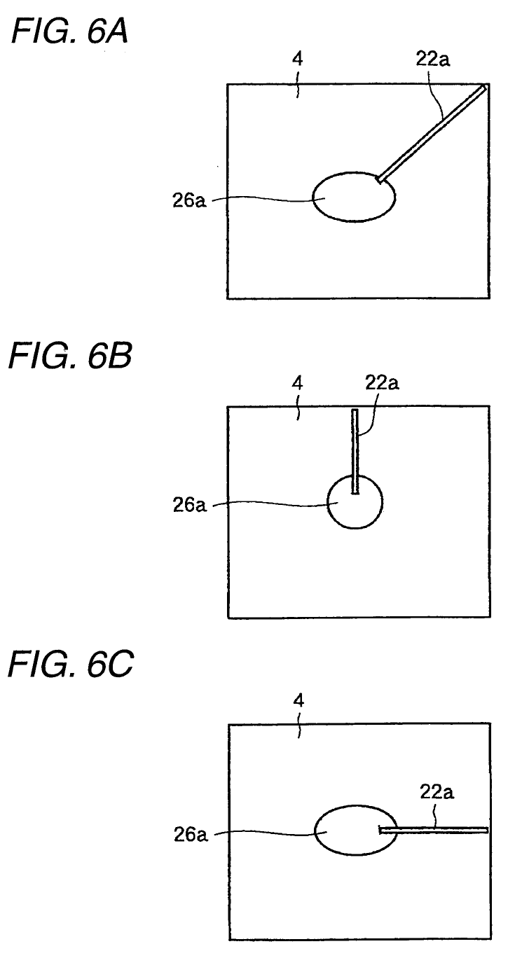

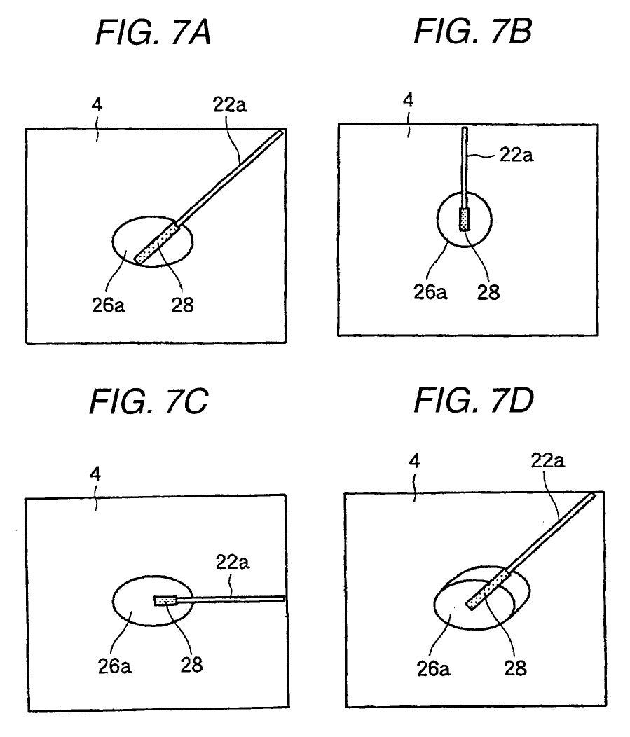

First, the puncture needle 22 is mounted to the inside of the puncture adaptor 21 of the probe 2. Subsequently, the ultrasonic diagnosis starts in such a manner that the front end portion of the probe 2 makes contact with a surface of the subject P, and the ultrasonic wave is three-dimensionally scanned. In terms of the ultrasonic diagnosis, the ultrasonic image is displayed on the monitor 4. When an image (3D image) 26a of the affected area 26 is observed as shown in FIG. 4, the puncture needle 22 is inserted from the body surface of the subject P toward the affected area 26 in the biological body while seeing the image 26a. The inserted puncture needle 22 is displayed in projection as an image 22a on the monitor 4 as shown in FIG. 5. Then, when the front end portion of the inserted puncture needle 22 arrives at a position in the vicinity of the affected area 26, a doctor manipulates the input device 3 to display the tomographic image obtained in the surface where the puncture needle 22 is inserted and the tomographic images obtained in the surfaces perpendicular to the puncture-needle-insertion surface in two directions shown in FIGS. 6A to 6C together with the 3D image 26a shown in FIG. 5. That is, the image 26a of the affected area 26 and the image 22a of the puncture needle 22 are respectively displayed on the monitor 4. Additionally, when the gun button of the puncture needle 22 is pressed in this state and the inner needle 24 protrudes from the outer needle 23, the tissue at the affected area 26 expected to be extracted is displayed as an image 28 shown in FIGS. 7A to 7D.

The doctor determines whether the desired tissue is expected to be extracted while seeing the image 28. In a case where it is determined that the desired tissue is expected to be extracted, the gun button of the puncture needle 22 is pressed. Accordingly, the inner needle 24 protrudes from the outer needle 23 to be inserted into the affected area 26 and to extract the tissue.

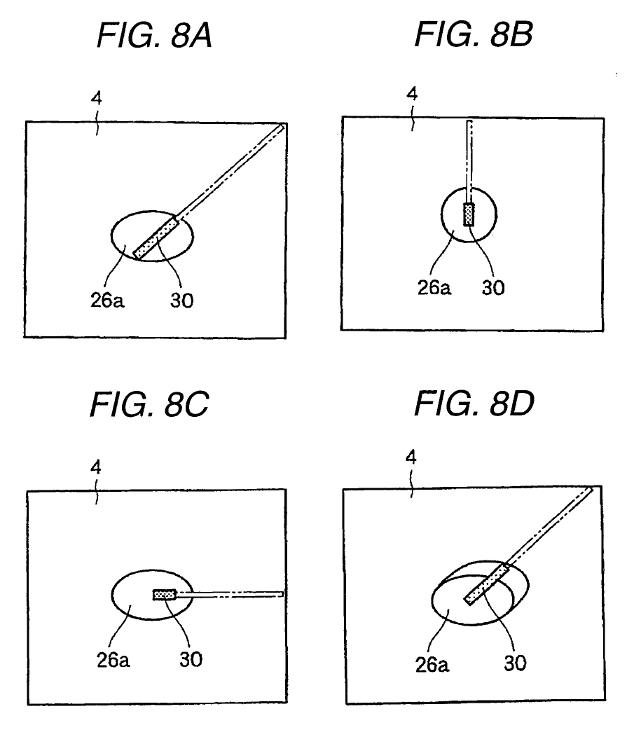

Likewise, when the tissue at the affected area 26 is extracted in actual, the extracted tissue is displayed on the monitor 4 in a form of an image 30 shown in FIGS. 8A to 8D, and the image 30 is stored as evidence for the actually extracted tissue.

Additionally, since the image 28 showing the tissue expected to be extracted substantially has the same shape as that of the image 30 showing the extracted tissue, the image color may be set to different from each other so as to accurately distinguish them.

Further, in a case where the position of the puncture needle 22 cannot be detected even when the puncture needle 22 is inserted from the body surface of the subject P toward the affected area 26 in the body, it is not possible to display the tissue expected to be extracted as the image.

As described above, according to this embodiment, when the gun button is pressed in a state where the affected area 26 is set to a target and the puncture needle 22 is inserted into a position in the vicinity thereof, since the tissue expected to be extracted is displayed as the image 28 on the monitor 4, the doctor is capable of determining whether the desired tissue can be extracted by seeing the image 28 before the extracting operation is carried out. Accordingly, it is possible to extract the desired tissue and to prevent the needle biopsy from being carried out two times.

Additionally, the movement of the needle is observed by means of an image recognition technology or a Doppler effect, and the image 30 of the tissue extracted by the needle biopsy is stored as evidence. Accordingly, in a case where the doctor wants to check later a portion where the tissue is actually extracted, it is possible to easily check the portion just by seeing the image 30. Thus, it is not necessary to meaninglessly carry out the needle biopsy again like the related art.

### Second Embodiment

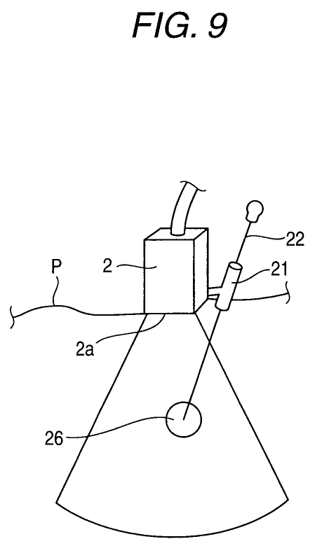

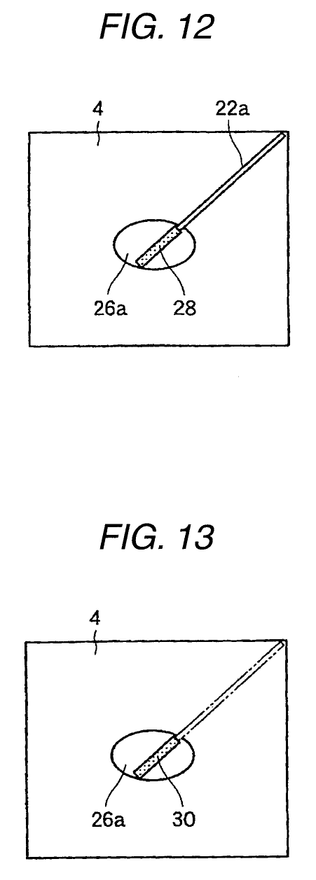

FIGS. 9 to 13 show a second embodiment of the invention.

Additionally, the same reference numerals are given to the same components as those described in the first embodiment, and the detailed description thereof will be omitted.

In the first embodiment, the needle biopsy is carried out in such a manner that the front end portion of the probe 2 makes contact with the surface of the subject P, and the ultrasonic wave is three-dimensionally scanned. However, in the second embodiment, as shown in FIG. 9, the needle biopsy is carried out in such a manner that the front end portion 2a of the probe 2 makes contact with the surface of the subject P, and the ultrasonic wave is two-dimensionally scanned.

Next, a case will be described in which the needle biopsy is carried out by two-dimensionally scanning the ultrasonic wave.

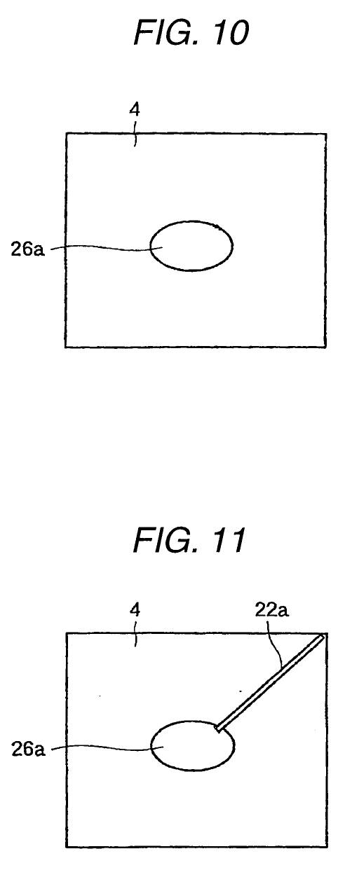

First, the puncture needle 22 is mounted to the inside of the puncture adaptor 21 of the probe 2. Subsequently, the ultrasonic diagnosis starts in such a manner that the front end portion 2a of the probe 2 makes contact with a surface of the subject P, and the ultrasonic wave is two-dimensionally scanned. In terms of the ultrasonic diagnosis, the ultrasonic image is displayed on the monitor 4. When an image 26a of the affected area 26 is observed as shown in FIG. 10, the puncture needle 22 is inserted from the body surface of the subject P toward the affected area 26 in the biological body while seeing the image 26a. The inserted puncture needle 22 is displayed as the image 22a in projection on the monitor 4 as shown in FIG. 11. Then, when the front end portion of the inserted puncture needle 22 arrives at a position in the vicinity of the affected area 26, the doctor manipulates the input device 3 to display the image 28 showing the inner needle 24 upon pressing the gun button of the puncture needle 22.

The doctor determines whether the desired tissue is expected to be extracted while seeing the image 28. In a case where it is determined that the desired tissue is expected to be extracted, the gun button of the puncture needle 22 is pressed. Accordingly, the inner needle 24 protrudes from the outer needle 23 to be inserted into the affected area 26 and to extract the tissue.

Likewise, when the tissue at the affected area 26 is extracted in actual, the extracted tissue is displayed on the monitor 4 in a form of an image 30 shown in FIG. 13, and the image 30 is stored as evidence for the actually extracted tissue.

Even in the second embodiment, it is possible to obtain the same effects and advantages as those of the first embodiment.

Additionally, in the first and second embodiments, after the tissue at the affected area 26 is extracted in actual, the image of the extracted tissue is stored as evidence. The image may be stored as a still image by freezing the image at the time immediately after the storage, for example, after 5 or 10 seconds.

Likewise, in a case where the image is stored as the still image by freezing the image, it is possible to easily obtain the image 30 of the extracted tissue and to prevent the storage of the unnecessary image.

Further, the present invention is not limited to the above-described embodiments, but the components may be modified in the scope without departing from the gist of the invention. Additionally, various inventions may be made through the appropriate combination of the plurality of components disclosed in the above-described embodiments. For example, several components may be omitted from all the components shown in the above-described embodiments. Then, the components shown in the different embodiments may be appropriately combined.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic diagnostic apparatus for two-dimensionally or three-dimensionally scanning a subject using an ultrasonic wave from an ultrasonic contact member and for assisting an operation for extracting a tissue of an extracted portion, detected by the ultrasonic scanning operation, using a puncture needle, the ultrasonic diagnostic apparatus comprising:
an image creating device configured to create an image of the extracted portion and an image of the puncture needle inserted into the subject by means of the ultrasonic scanning operation; and
a display device configured to display the image of the extracted portion and the image of the puncture needle created by the image creating device,
wherein the image creating device displays a tissue expected to be extracted from the extracted portion as an image on the display device before the puncture needle is inserted into the extracted portion.

2. The ultrasonic diagnostic apparatus according to claim 1, wherein the image creating device displays the extracted portion as a three-dimension image on the display device, displays a tomographic image in a surface where the puncture needle is inserted and tomographic images in surfaces perpendicular to the puncture-needle-insertion surface in two directions on the display device, and then displays an image of the tissue expected to be extracted on each displayed surface.

3. The ultrasonic diagnostic apparatus according to claim 1, wherein the ultrasonic contact member includes a puncture adaptor used to insert the puncture needle, and
wherein the puncture adaptor is provided with a position sensor or a rotary roller, and a position is recognized by measuring an inserted amount of the puncture needle by means of the position sensor or the rotary roller.

4. An ultrasonic diagnostic apparatus for two-dimensionally or three-dimensionally scanning a subject using an ultrasonic wave from an ultrasonic contact member and for assisting an operation for extracting a tissue of an extracted portion, detected by the ultrasonic scanning operation, using a puncture needle, the ultrasonic diagnostic apparatus comprising:
an image creating device configured to create an image of the extracted portion and an image of the puncture needle inserted into the subject by means of the ultrasonic scanning operation; and
a display device configured to display the image of the extracted portion and the image of the puncture needle created by the image creating device,
wherein the image creating device displays the extracted tissue as an image on the display device and stores the image as evidence after the tissue of the extracted portion is extracted by the puncture needle.

5. The ultrasonic diagnostic apparatus according to claim 4, wherein the image creating device freezes the image to store the image immediately after the tissue of the extracted portion is extracted by the puncture needle.

6. The ultrasonic diagnostic apparatus according to claim 4, wherein the image creating device displays the extracted portion as a three-dimension image on the display device, displays a tomographic image in a surface where the puncture needle is inserted and tomographic images in surfaces perpendicular to the puncture-needle-insertion surface in two directions on the display device, and then displays the extracted tissue as an image on each displayed surface to be stored as evidence after the tissue of the extracted portion is extracted.

7. The ultrasonic diagnostic apparatus according to claim 4, wherein the ultrasonic contact member includes a puncture adaptor used to insert the puncture needle, and
wherein the puncture adaptor is provided with a position sensor or a rotary roller, and a position is recognized by measuring an inserted amount of the puncture needle by means of the position sensor or the rotary roller.
